(19) United States
(12) Patent Application Publication
(10) Pub. No.: US 2025/0114222 A1
(43) Pub. Date: Apr. 10, 2025

(54) BIODEGRADABLE MOLYBDENUM-BASED FLOW DIVERTER AND PREPARATION METHOD THEREOF

(71) Applicant: SOUTHWEST JIAOTONG UNIVERSITY, Chengdu (CN)

(72) Inventors: GUOJIANG WAN, Chengdu (CN); FEI GAO, Chengdu (CN); YUKUN ZHOU, Chengdu (CN); JINJING LIU, Chengdu (CN); WENJIE TAO, Chengdu (CN); ZHAOZHAO ZHANG, Chengdu (CN)

(21) Appl. No.: 18/829,403

(22) Filed: Sep. 10, 2024

Related U.S. Application Data
(63) Continuation of application No. PCT/CN2024/104639, filed on Jul. 10, 2024.

(30) Foreign Application Priority Data
Oct. 10, 2023 (CN) 202311311237.7

(51) Int. Cl. A61F 2/90; A61F 2/82; A61L 31/02; A61L 31/14; B21F 45/00

(52) U.S. Cl. CPC A61F 2/90; A61L 31/022; A61L 31/148; A61F 2002/823; B21F 45/008

(57) ABSTRACT

The present invention discloses a biodegradable molybdenum-based flow diverter and a preparation method thereof, wherein the preparation method comprises the following steps: step 1. pretreatment of a molybdenum wire: cleaning and drying the molybdenum wire, and then weaving to obtain the molybdenum-based flow diverter; step 2. performing heat treatment on the molybdenum-based flow diverter in the step 1; and step 3. performing surface chemical polishing treatment on the molybdenum-based flow diverter subjected to the heat treatment in the step 2. The present invention solves the technical problem that the flow diverter in the prior art cannot be degraded in vivo and therefore easily causing various complications. It provides also improved mechanical property, MRI magnetic resonance imaging (MRI) compatibility as well as biocompatibility, and particularly has the function of promoting rapid endothelialization, compared with other degradable materials.

### CROSS-REFERENCE TO RELATED APPLICATIONS

This application is a continuation of co-pending International Patent Application No. PCT/CN2024/104639, filed on Jul. 10, 2024, which claims the priority and benefit of Chinese patent application number 202311311237.7, filed on Oct. 10, 2023 with China National Intellectual Property Administration, the entire contents of which are incorporated herein by reference.

### TECHNICAL FIELD

The present invention relates to the technical field of medical instruments, and in particular, to a biodegradable molybdenum-based flow diverter and a preparation method thereof.

### BACKGROUND

An intracranial aneurysm (IA) refers to outward ballooning of weakened arterial walls in a cerebrovascular system, and has a prevalence rate of 3.2% in the 40-60 years old population. The unruptured intracranial aneurysm is the most dangerous because the intracranial aneurysm may rupture at any time and cause subarachnoid hemorrhage and related complications, which are usually fatal. A flow diverter is a densely meshed metal stent, which acts to redirect a large portion of the blood flowing inside the aneurysm, promotes thrombus formation within the aneurysm, and eventually leads to aneurysm occlusion. This method can effectively reduce the negative effects of traditional surgical clipping and coil embolization, and achieve better treatment efficacy. However, most of the flow diverters currently used in clinical practice are made of nickel-titanium alloy or cobalt-chromium alloy. The stents made of such materials will be permanently present in the patient's body and may not even be removed by a secondary surgery, which inevitably leads to the risk of related complications. In addition, although such alloys have good biocompatibility, they fall short of biological functionality to promote rapid endothelialization for expediting vascular remodeling.

A biodegradable flow diverter is expected to solve the above problems. An ideal biodegradable flow diverter can occlude and heal an aneurysm and can be gradually absorbed by the body, thus eliminating the complications associated with the permanent presence of the stent. Biodegradable materials are mainly divided into two categories: polymer and metal. Although polymer materials have good degradability and biosafety, their weak mechanical properties make it impossible to use them in most medical situations. For biodegradable metals, the existing research mainly focuses on iron, magnesium, and zinc. However, the slower degradation rate of iron and the insoluble corrosion products, the insufficient mechanical properties of magnesium and the generation of hydrogen during corrosion, and the poor corrosion mode and cytotoxicity of zinc all limit the application of these materials in biodegradable materials. Moreover, due to intrinsic properties of these materials, it is extremely difficult to prepare them into an ultra-fine wire with good mechanical properties for the weaving of flow diverters.

### SUMMARY

Aiming at addressing the defects of the above biodegradable metals, an objective of the present invention is to provide a biodegradable molybdenum-based flow diverter, which can possess outstanding mechanical properties, have a proper corrosion rate as well as a relatively uniform corrosion mode in simulated body fluid immersion. The molybdenum-based flow diverters are expected to degrade gradationally in their clinical service and maintain the integrity of structural support to the maximum extent. Uniform corrosion, material surface oxides and special active metal ions are all conducive to the adhesion and proliferation of endothelial cells on the surface of the densely meshed stent, so that the densely meshed stent has a tendency of faster endothelialization and therefore better biocompatibility.

The present invention adopts the following technical solutions.

A preparation method of a biodegradable molybdenum-based flow diverter comprises the following steps:
step 1. pretreatment of a molybdenum wire: cleaning and drying the molybdenum wire, and then weaving to obtain the molybdenum-based flow diverter;
step 2. performing heat treatment on the molybdenum-based flow diverter in the step 1; and
step 3. performing surface chemical polishing treatment on the molybdenum-based flow diverter subjected to the heat treatment in the step 2.

Further, in the step 2, the heat treatment is implemented by keeping the temperature at 510° C. for 8 minutes and then cooling in pure water for 1 minute.

Further, in the step 3, the surface chemical polishing treatment is implemented by placing the diverter in 3% HCl at 50° C. for 1 minute, then taking the diverter out, sequentially washing the diverter with distilled water and pure ethanol for 1 time under ultrasonic conditions, then placing the diverter in 3% HCl at 50° C. again, and repeating the washing for 10 times.

Another aspect of the present invention provides a biodegradable molybdenum-based flow diverter, wherein a side wall of the diverter is woven with a molybdenum wire in a staggered fashion to form a densely meshed structure, an outer cylindrical diameter of the flow diverter is 2-5 mm, and an overall metal coverage rate of a degradable molybdenum wire woven stent is 30-35%.

Further, the degradable molybdenum wire has a purity of higher than 99.9% and a diameter of smaller than 0.035 mm;
the degradable molybdenum wire has a tensile strength of higher than 1800 MPa; and
the degradable molybdenum wire has an elongation of higher than 3%.

The beneficial effects of the present invention are as follows.

1. The most important beneficial effect of the present invention is that the flow diverter is woven with a biodegradable molybdenum wire. The flow diverter woven with the biodegradable material can effectively solve many problems of the existing non-degradable flow diverter. In particular, among the candidates for the degradable metal materials, the existing research mainly focuses on iron, magnesium, and zinc. However, the slower degradation rate of iron and the insoluble corrosion products, the insufficient mechanical properties of magnesium and the generation of hydrogen during corrosion, and the poor corrosion mode and cytotoxicity of zinc all limit the application of these materials in biodegradable materials. Moreover, due to the insufficient mechanical properties of these materials, it is difficult to prepare filaments with qualified mechanical properties, and it is even more difficult to make the filament diameter of these materials smaller than 0.035 mm. The better mechanical properties of molybdenum wire and the mature wire manufacturing process enable the potential of the molybdenum wire to be far higher than that of the existing iron, magnesium and zinc.

2. Molybdenum ions and surface products thereof released in the degradation process of molybdenum have a certain ability to promote the adhesion and proliferation of endothelial cells, and the good uniform corrosion mode of the molybdenum also enables endothelial cells to be more densely adhered, so that new vessel walls can be generated outside the flow diverter as soon as possible, and the purpose of completely blocking unruptured intracranial aneurysms is achieved. In this way, even if the molybdenum-based flow diverter is completely degraded after the end of service, there will be no secondary attack due to incomplete occlusion of unruptured intracranial aneurysms.

3. Molybdenum has better MRI developing property, so that the molybdenum-based flow diverter can have clear images in the implantation process without adding a non-degradable Pt developing wire in the weaving process, and the complete flow diverter is really prepared by completely degradable materials. However, the existing flow diverters cannot simultaneously meet the above properties.

4. The side wall of the diverter is woven with a molybdenum wire in a staggered fashion to form a densely meshed structure, and the metal coverage rate of the flow diverter is 30-35%. The high metal coverage rate can better change the direction of blood flow in blood vessels and stop the blood from exchanging with the unruptured intracranial aneurysm. The outer cylindrical diameter of the flow diverter is 2-5 mm, and the diameter specification can be used for most of the neurovascular vessels

5. The beneficial synergy after heat treatment of the present invention is to improve the flexibility and the support of the molybdenum-based flow diverter, the heat treatment can lead the surface of the material covered with part of the oxide in advance, and the molybdenum can be degraded more uniformly according to the corrosion degradation mechanism of the molybdenum.

### BRIEF DESCRIPTION OF DRAWINGS

To more clearly illustrate the technical solutions of the examples of the present invention, the accompanying drawings of the examples are briefly introduced below. Apparently, the accompanying drawings in the following descriptions only relate to some examples of the present invention, but are not intended to limit the present invention.

FIG. 1 is a schematic diagram of metal coverage rate calculation according to the present invention;

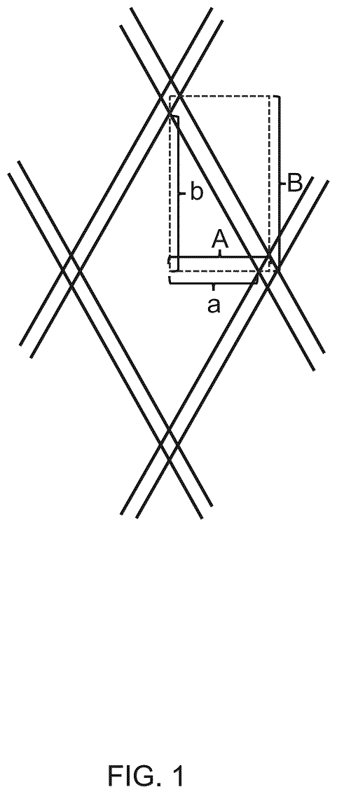

FIG. 2 is a schematic diagram of a surface morphology of a molybdenum-based flow diverter according to the present invention;

FIG. 3 is a schematic diagram of corrosion morphology and a pH value during immersion of a molybdenum-based flow diverter in Hank's solution according to the present invention;

FIG. 4 is a schematic diagram of EDS analysis of corrosion products of a molybdenum-based flow diverter in Hank's solution according to the present invention;

FIG. 5 is a schematic diagram of XRD and XPS of corrosion products of a molybdenum-based flow diverter in Hank's solution according to the present invention;

FIG. 6 is a schematic diagram of a morphology of a molybdenum-based flow diverter after immersion in Hank's solution with corrosion products removed according to the present invention and a corrosion rate calculated by a weight loss method;

FIG. 7 is a schematic diagram of direct culture of endothelial cells on a surface of a molybdenum-based flow diverter according to the present invention and CCK-8 detection; and

FIG. 8 shows blood compatibility of a molybdenum-based flow diverter according to the present invention.

### DETAILED DESCRIPTION OF THE EMBODIMENTS

To make objectives, technical solutions, and advantages of examples of the present invention clearer, the following clearly and completely describes technical solutions in examples of the present invention with reference to accompanying drawings in examples of the present invention. It is clear that the described examples are some but not all of examples of the present invention. Based on the described examples of the present invention, all other examples obtained by those of ordinary skill in the art without creative efforts fall within the protection scope of the present invention.

The present invention is further described below with reference to the accompanying drawings and examples.

A preparation method of a biodegradable molybdenum-based flow diverter comprises the following steps:

Step 1. pretreatment of a molybdenum wire: cleaning and drying the molybdenum wire, and then weaving to obtain the molybdenum-based flow diverter.

Step 2. performing heat treatment on the molybdenum-based flow diverter in the step 1.

Specifically, the heat treatment is implemented by keeping the temperature at 510° C. for 8 minutes, taking out a stent after the timing is over and placing the stent in pure water to cool for 1 minute.

Step 3. performing surface chemical polishing treatment on the molybdenum-based flow diverter subjected to the heat treatment in the step 2.

Specifically, in the step 3, the surface chemical polishing treatment is implemented by placing the diverter in 3% HCl at 50° C. for 1 minute, then taking the diverter out, sequentially washing the diverter with distilled water and pure ethanol for 1 time under ultrasonic conditions, then placing the diverter in 3% HCl at 50° C. again, and repeating the washing for 10 times.

A biodegradable molybdenum-based flow diverter is prepared by the above preparation method, wherein a side wall of the diverter is woven with a molybdenum wire in a staggered fashion to form a densely meshed structure, an outer cylindrical diameter of the flow diverter is 3 mm, and an overall metal coverage rate of a degradable molybdenum wire woven stent is 30-35%. The metal coverage rate is calculated as follows: the stent is placed in a rigid transparent tube, and the values of a, b, A and B in the figure are measured as follows. The metal coverage rate is 100%−ab/AB*100%. Specifically, the degradable molybdenum wire has a purity of higher than 99.9% and a diameter of smaller than 0.035 mm; the degradable molybdenum wire has a tensile strength of higher than 1800 MPa; and the degradable molybdenum wire has an elongation of higher than 3%.

### Example 1

Example 1 provides a biodegradable molybdenum-based flow diverter and a preparation method thereof. The method comprises:

Step 1. pretreatment of a molybdenum wire: placing the molybdenum wire with a diameter of 0.027 mm and a purity of 99.95% in deionized water and pure ethanol, sequentially cleaning under an ultrasonic condition, and weaving the molybdenum wire after drying without adding a developing wire in the weaving process.

Step 2. heat treatment of the molybdenum-based flow diverter: putting the molybdenum-based flow diverter into a muffle furnace for heat treatment of the material, wherein the heat treatment is implemented by keeping the temperature at 510° C. for 8 minutes, taking out a stent after the timing is over and placing the stent in pure water to cool for 1 minute.

Step 3. surface chemical polishing of the molybdenum-based flow diverter, wherein the surface chemical polishing treatment is implemented by placing the diverter in 3% HCl at 50° C. for 1 minute, then taking the diverter out, sequentially washing the diverter with distilled water and pure ethanol for 1 time under ultrasonic conditions, then placing the diverter in 3% HCl at 50° C. again, and repeating the washing for 10 times.

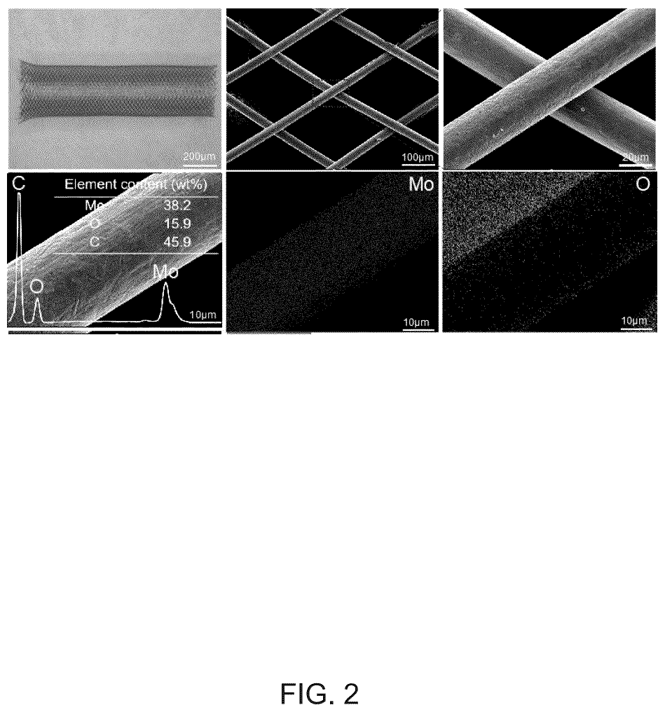

The surface morphology of the molybdenum-based flow diverter woven in Example 1 is shown in FIG. 2, which shows that the surface of the molybdenum-based flow diverter after weaving and cleaning is provided with a neat densely meshed structure, and no impurities are provided on the surface. EDS analysis of the surface of the molybdenum-based flow diverter also confirms that the filaments are mainly made of molybdenum and oxygen (it should be noted that the higher carbon content in the element is because the sample is a wire mesh structure, which inevitably obtains stronger carbon information in the back-side conductive adhesive).

### Experimental Example 1

This example evaluated the corrosion degradation performance and mechanism of the molybdenum-based flow diverter prepared in Example 1 by long-term in vitro immersion. The exposed surface area of the flow diverter was calculated to be 0.641 cm² based on the metal coverage rate, Hank's solution was used as the simulated body fluid, and a long-term immersion for 28 days was performed according to a solution volume to sample surface area ratio standard of 20 mL/cm² during which pH measurements and simulated body fluid changes were performed every three days. The specific preparation proportion of the Hank's solution was as follows: 8.00 g/L NaCl, 0.40 g/L KCl, 0.10 g/L MgCl, 0.35 g/L NaHCO₃, 0.06 g/L MgSO₄·7H₂O, 0.14 g/L CaCl₂, 0.06 g/L Na₂HPO₄·12H₂O, 0.06 g/L KH₂PO₄, 1.00 g/L C₆H₁₂O₆ and deionized water, and the pH was adjusted to about 7.4 by using 3.6% NaHCO₃ solution.

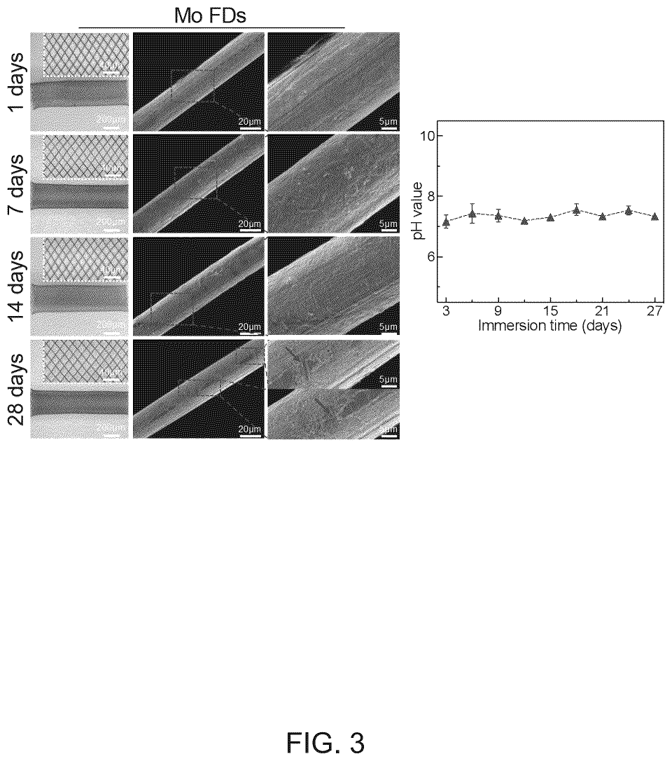

FIG. 3 shows a macroscopic stereomicroscope photograph and a microscopic scanning electron microscopy photograph with the sample taken out. As shown in FIG. 3, in the immersion process, the molybdenum-based flow diverter macroscopically shows that the surface color of the stent gradually deepens along with the increase of the immersion time. It can be observed from the microscopic images that corrosion products appears on the surface of the stent in the early stage of corrosion (7 days), corrosion cracks perpendicular to the wire stretching direction appears on the surface of the diverter on the day 14, and the number of such cracks gradually increases and the width of these cracks also gradually widens on the day 28. Through the analysis of the microscopic corrosion morphology, it can be concluded that the molybdenum-based flow diverter has certain degradability. It can also be seen from the pH value that, in the 28-day long-term in vitro corrosion experiment, the molybdenum-based flow diverter has little effect on the pH of the environment and remains stable throughout the process within a pH range suitable for various reactions in the body.

### Experimental Example 2

Based on Experimental Example 1, this example further performed elemental analysis on the surface products of the molybdenum-based flow diverter after corrosion in vitro in Hank's solution.

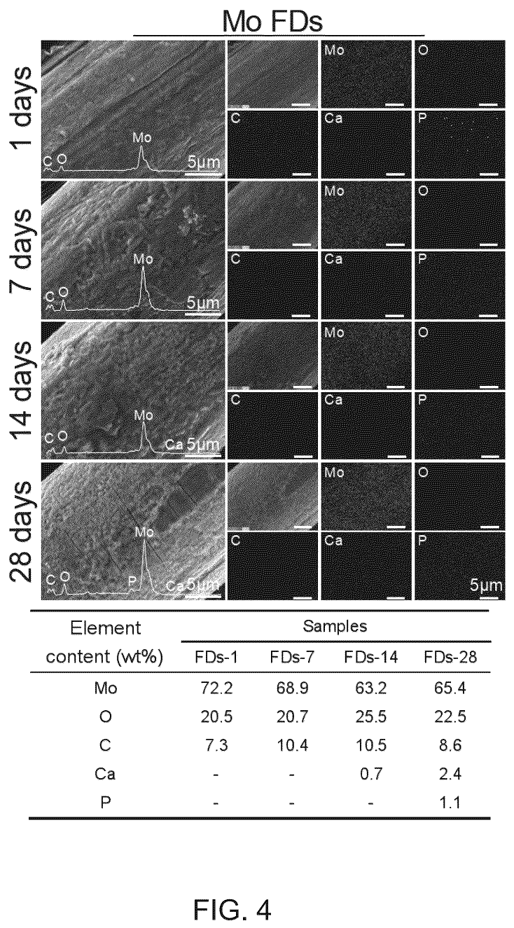

FIG. 4 shows an elemental analysis of corrosion products of a molybdenum-based flow diverter in Hank's solution. As shown in FIG. 4, it can be seen from the element spectrum and element content that the corrosion products on the surface of the molybdenum-based flow diverter are mostly molybdenum oxides. It should be noted that, in the middle and later stages of corrosion, Ca and P elements are also detected on the surface of the molybdenum-based flow diverter, which may be caused by immersion in Hank's solution, and some precipitates are formed on the surface of the molybdenum-based flow diverter, but have a small content, so that the molybdenum-based flow diverter placed in a real blood vessel environment does not hinder blood flow due to the accumulation of calcium phosphate salts.

### Experimental Example 3

Based on Experimental Example 2, this example further performed XRD and XPS analysis on the surface corrosion products of the molybdenum-based flow diverter.

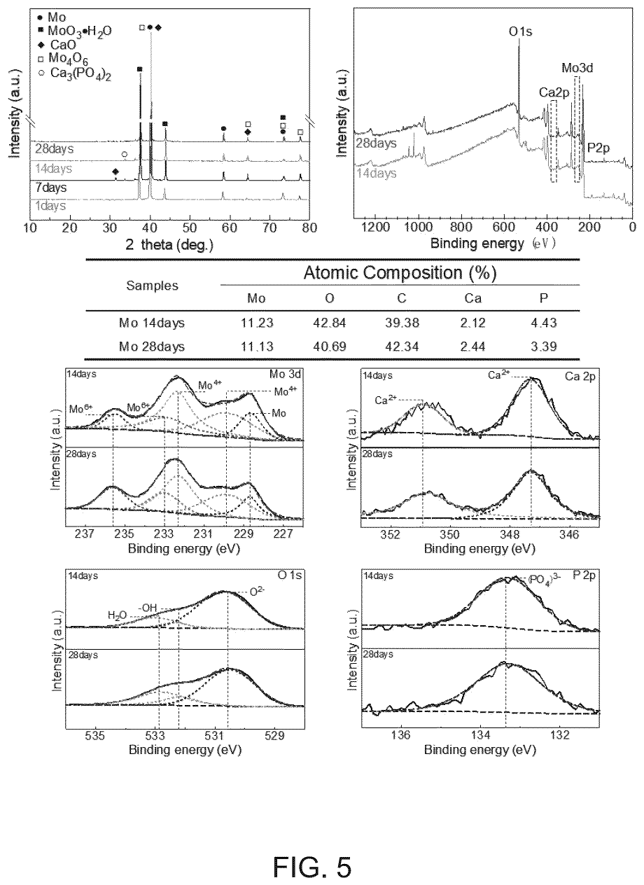

FIG. 5 shows XRD and XPS of corrosion products of a molybdenum-based flow diverter in Hank's solution. As shown in FIG. 5, it can be observed from the X-ray diffraction pattern that most of the products on the surface of the material are oxides and hydrates of molybdenum at different valences. Calcium phosphate salts are also observed in some special places, but have a very weak intensity. The samples were then subjected to X-ray photoelectron spectroscopy testing, and it can be seen from the peak analysis of the results that the valence state of molybdenum oxide gradually increased in the corrosion process.

### Experimental Example 4

Based on Experimental Example 2, this example removes corrosion products from the molybdenum-based flow diverter. Specifically, a 200 g/L CrO₃ solution was used, the flow diverter was completely immersed in the solution at 80° C., and the sample was taken out after waiting for 5 minutes. 4 parallel samples were selected in each group to remove corrosion products, which was used to calculate the corrosion rate of molybdenum-based flow diverter by a weight loss method. Specifically, the weight of the samples before immersion and after removal of corrosion products was measured to calculate the corrosion rate (CR) in mm/year. A calculation formula is as follows:

$$C R = ( K \times W ) / ( A \times T \times D )$$

wherein K is a constant (8.76×10⁴); W is the mass difference of the sample before and after immersion in g; A is the area of the sample exposed to the corrosive medium in cm²; T is the immersion time in h; and D is the density in g/cm³.

After the sample was taken out from the CrO₃ solution, the sample was cleaned with deionized water and anhydrous ethanol under ultrasonic conditions for 3 cycles, and finally the surface was blown dry.

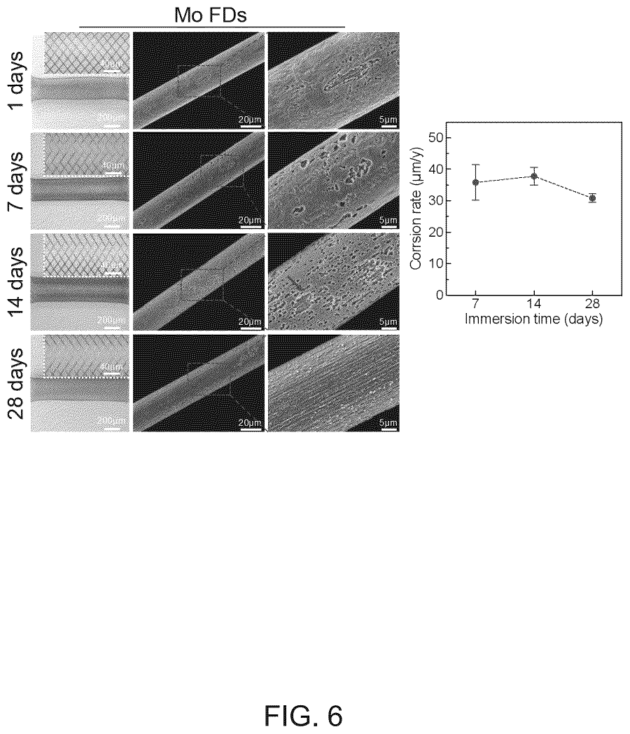

FIG. 6 shows a morphology of a molybdenum-based flow diverter after immersion in Hank's solution with corrosion products removed and a corrosion rate calculated by a weight loss method. As shown in FIG. 6, it can be seen from the scanning electron microscopy images that, in the initial stage of corrosion, local corrosion products on the surface of the molybdenum-based flow diverter fall off or dissolve. These local corrosion surfaces gradually expand and eventually connect over time. Until the day 28, the first layer of corrosion products on the surface have completely fallen off. Combined with the analysis of Experimental Example 4, the corrosion products of the molybdenum-based flow diverter can initially form low-valence molybdenum oxides in the corrosion process, the molybdenum oxides can gradually transform into high-valent ones with the increase of the corrosion time, and gradually fall off or dissolve in the corrosion process, revealing a fresher molybdenum matrix at the bottom. The exposed molybdenum can continuously follow this rule to generate oxides, so that the molybdenum has a degradation mode of falling off layer by layer. The corrosion rate calculated by the weight loss method shows that the corrosion rate of the molybdenum-based flow diverter is about 30-40 m/year.

### Experimental Example 5

In this example, endothelial cells were directly cultured in vitro using the molybdenum-based flow diverter prepared in Example 1, and the biocompatibility of the molybdenum-based flow diverter was evaluated by comparing with the existing nickel-titanium stent widely used in clinical practice.

The cells used in this experiment were human umbilical vein endothelial cells (HUVECs), which were cultured in a-MEM medium containing 10% fetal bovine serum and 1% penicillin/streptomycin in a cell incubator at 37° C. with a 5% CO₂ concentration.

Direct culture method: the molybdenum-based flow diverter in Example 1 was sterilized by ultraviolet and then placed in a 24-well plate. HUVEC cells with a cell density of 2×10⁴ cell/mL were inoculated on the sample surface, and CCK-8 activity test and rhodamine staining were performed after culturing for 1, 3, and 5 days, respectively. After the fluorescence photography, the molybdenum-based flow diverter was dehydrated. Specifically, the samples were placed in 50%, 60%, 70%, 80%, 90%, and 100% anhydrous ethanol (the balance was deionized water) for 15 minutes, and then gradient dehydrated and sprayed with gold for scanning electron microscopy photography. It should be noted that the samples from the last day were exchanged when the culture reached the third day.

CCK-8 activity test: the cell culture medium was aspirated, and an equal amount of 10% CCK-8 culture medium was added and cultured in the dark for 2-4 hours. Then, 100 L of the supernatant was taken out and transferred to a new 96-well plate, and the OD value at 450 nm was measured using a microplate reader.

Rhodamine staining: after the CCK-8 activity test was completed, the liquid was aspirated, washed 3 times with PBS solution, and fixed for 3 hours with 2.5% glutaraldehyde; then the glutaraldehyde was aspirated, and washed three times with PBS solution; finally, 100 L of rhodamine was added to each well for staining for 8 minutes, and then the cell morphology was observed using a fluorescence microscope.

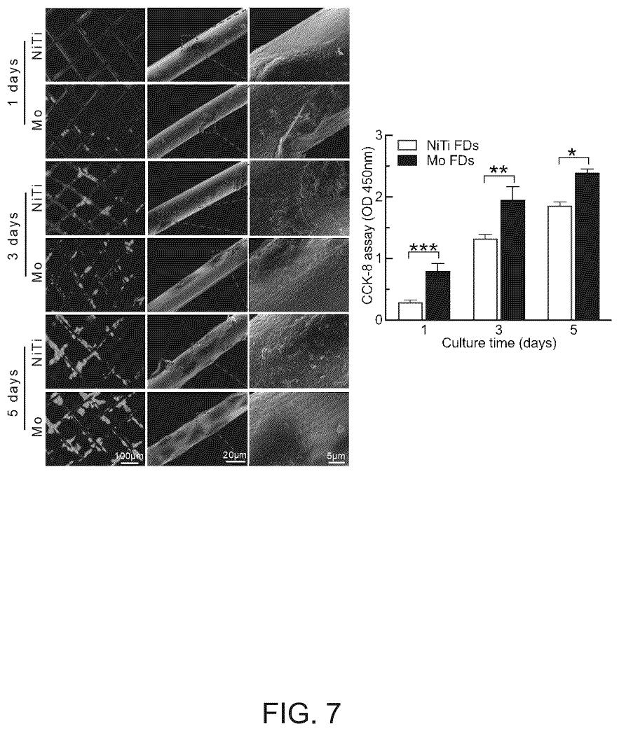

FIG. 7 shows fluorescence, scanning electron microscopy images and CCK-8 detection of endothelial cells cultured directly on the surface of the molybdenum-based flow diverter. As shown in FIG. 7, endothelial cells adhered to the molybdenum-based flow diverter after one day of culture, as the culture time increased to 3 and 5 days, it can be seen that the cells gradually proliferated on the stent, and finally almost covered the entire stent on the fifth day. It can also be seen from the scanning electron microscopy images with higher magnification that the endothelial cells on the molybdenum-based flow diverter well spread on the fifth day. Although the nickel-titanium densely meshed stent that has been used clinically also has good biocompatibility, the number of endothelial cells of this densely meshed stent is smaller than that of the molybdenum-based flow diverter. The CCK-8 results also show a similar trend, and the cell activity of the molybdenum-based flow diverter is higher than that of the traditional nickel-titanium stent at three time points, which indicates that the molybdenum ions released by the molybdenum-based flow diverter in the degradation process can effectively promote the proliferation and adhesion of endothelial cells, and the molybdenum-based flow diverter has more potential in biological functionality.

### Experimental Example 6

In this example, three blood coagulation items and hemolysis rate were performed using the molybdenum-based flow diverter prepared in Example 1 to evaluate the performance of the molybdenum-based flow diverter in blood compatibility.

The three blood coagulation items comprise APTT, TT and PT, and the specific operation is as follows: fresh blood containing 0.02 g/mL of anticoagulant sodium oxalate was centrifuged at 3000 rpm for 15 min to obtain platelet-poor plasma (PPP) for subsequent experiments. 200 μL of PPP and 100 μL of actin-activated brain activin reagent mixture were added to the sample surface, and then 100 μL of CaCl₂ (0.03 mol/L) was added. After incubation at 37° C. for 30 min, APTT was measured; 200 μL of PPP was dropped onto the sample surface and incubated at 37° C. for 30 min. Then, 200 μL of incubated PPP solution and 200 μL of TT reagent were added to the test tube for TT assay; and in PT assay, 200 μL of incubated PPP solution was added to 100 μL of PT reagent for assay.

Hemolysis rate: the molybdenum-based flow diverter was immersed in normal saline at 37° C. at a ratio of 0.5 g/mL and incubated for 30 min to obtain sample extracts. After 30 minutes, 200 μL of fresh blood diluted with normal saline was aspirated into 10 mL of the extract, wherein the volume ratio of blood to the normal saline was 4:5, and then the mixture was incubated at 37° C. for 1 hour. After the incubation time was over, the liquid was transferred to a new centrifuge tube and centrifuged at 1000 rpm for 5 min. 200 μL of the supernatant was pipetted into a 96-well plate, and the absorbance (545 nm) of the supernatant was measured using a microplate reader to calculate the hemolysis rate. Throughout the experiment, the normal saline was used as the negative control, and the distilled water was used as the positive control.

$$Hemolysis ( \% ) = A 1 - B 2 / B 1 - B 2 \times 100 \%$$

wherein A1 is the absorbance of the sample, B1 and B2 are the absorbance of the positive control group and the negative control group, respectively. All samples had at least four replicates to ensure statistical significance.

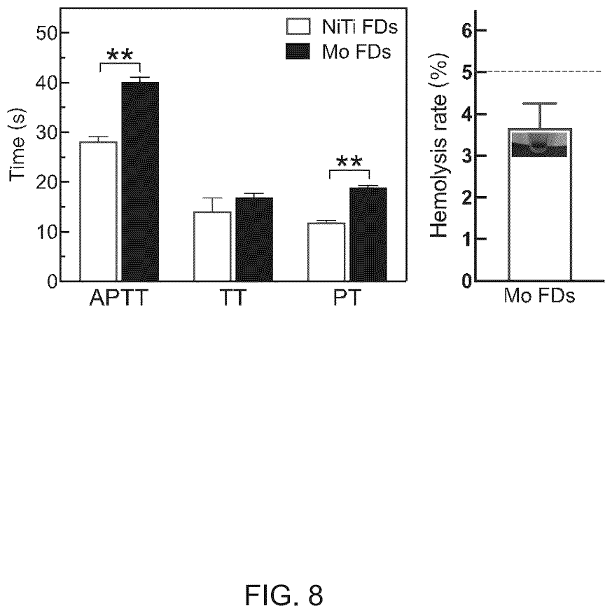

FIG. 8 shows blood compatibility of a molybdenum-based flow diverter. As shown in FIG. 8, in the experiment of three blood coagulation items, APTT and PT times are significantly higher than those of the existing clinical nickel-titanium flow diverter, which indicates that the molybdenum-based flow diverter has better anticoagulant functionality. The hemolysis rate of the molybdenum-based flow diverter is also smaller than 5%, meeting the safety standards for implants.

The above descriptions are only preferred examples of the present invention, and are not intended to limit the present invention in any form. Although the preferred examples above have disclosed the present invention, they are not intended to limit the present invention. Any of those familiar with the technical field, without departing from the scope of the technical solutions of the present invention, can use the technical content disclosed above to make various changes and modify the technical content as equivalent changes of the equivalent examples. However, any simple modifications, equivalent changes and modifications made to the above examples according to the technical spirit of the present invention without departing from the content of the technical solutions of the present invention shall fall within the scope of the technical solutions of the present invention.

What is claimed is:

1. A preparation method of a biodegradable molybdenum-based flow diverter, comprising the following steps:
step 1. pretreatment of a molybdenum wire: cleaning and drying the molybdenum wire, and then weaving to obtain the molybdenum-based flow diverter;
step 2. performing heat treatment on the molybdenum-based flow diverter in the step 1; wherein the heat treatment is implemented by keeping the temperature at 510° C. for 8 minutes and placing the flow diverter in pure water to cool for 1 minute; and
step 3. performing surface chemical polishing treatment on the molybdenum-based flow diverter subjected to the heat treatment in the step 2.

2. The preparation method of the biodegradable molybdenum-based flow diverter according to claim 1, wherein in the step 3, the surface chemical polishing treatment is implemented by placing the diverter in 3% HCl at 50° C. for 1 minute, then taking the diverter out, sequentially washing the diverter with distilled water and pure ethanol for 1 time under ultrasonic conditions, then placing the diverter in 3% HCl at 50° C. again, and repeating the washing for 10 times.

3. The preparation method of the biodegradable molybdenum-based flow diverter according to claim 1, wherein a side wall of the molybdenum-based flow diverter is woven with a molybdenum wire in a staggered fashion to form a densely meshed structure, an outer cylindrical diameter of the molybdenum-based flow diverter is 2-5 mm, and an overall metal coverage rate of the molybdenum wire woven stent is 30-35%.

4. The preparation method of the biodegradable molybdenum-based flow diverter according to claim 3, wherein the molybdenum wire has a purity of higher than 99.9% and a diameter of smaller than 0.035 mm;
the molybdenum wire has a tensile strength of higher than 1800 MPa; and
the molybdenum wire has an elongation of higher than 3%.
